(19) United States
(12) Patent Application Publication
(10) Pub. No.: US 2014/0377872 A1
(43) Pub. Date: Dec. 25, 2014

(54) METHOD OF PREDICTING THE AMOUNT AND THE COMPOSITION OF FLUIDS PRODUCED BY MINERAL REACTIONS OPERATING WITHIN A SEDIMENTARY BASIN

(71) Applicants: TOTAL SA, Courbevoie (FR); IFP Energies nouvelles, Rueil-Malmaison Cedex (FR)

(72) Inventors: Etienne BROSSE, MARLY LE ROI (FR); Jean-Luc RUDKIEWICZ, ANTONY (FR); Teddy PARRA, PARIS (FR); Xavier GUICHET, RUEIL-MALMAISON (FR)

(21) Appl. No.: 14/308,788

(22) Filed: Jun. 19, 2014

(30) Foreign Application Priority Data
Jun. 20, 2013 (FR) 1355860

(51) Int. Cl. G01N 33/24; G01V 9/00

(52) U.S. Cl. CPC G01N 33/24; G01V 9/00; G01N 33/246; G01N 33/241
USPC 436/29; 436/25; 702/11

(57) ABSTRACT

The invention is a method of predicting the amount and the composition of fluids produced by mineral reactions operating within a sedimentary basin and trapped with hydrocarbons in reservoirs. Geological data characteristic of the basin are acquired and a representation of the basin by a grid is constructed. The evolution of a depth of burial (z), a temperature (T), a pore pressure (P), a volume (V) and a porosity φ at successive ages (tᵢ) representative of the geological history of the basin is then calculated for at least one set of cells of the grid, using a basin model and the geological data. A mineralogical or chemical rock composition is determined in each cell of the set of cells from the geological data of the basin. The amount and the composition of fluids of mineral origin is determined within the set of cells using a geochemical model and an equation of state, from the parameters, the composition and a thermodynamic database.

### CROSS REFERENCE TO RELATED APPLICATION

Reference is made to French Patent Application Serial No. 1355.860, filed Jun. 20, 2013, which application is incorporated herein by reference in its entirety.

### BACKGROUND OF THE INVENTION

1. Field of the Invention

The present invention relates to the petroleum industry and more particularly to hydrocarbon exploration in sedimentary basins. In particular, the invention relates to a method allowing calculation of the amount and the composition of fluids of mineral origin generated by the reactions that occur among the minerals of sedimentary rocks as they are buried during the geological history of a basin. These fluids, characterized by high water (H₂O) and/or carbon dioxide (CO₂) content, typically form above a temperature (T) of 250° C. and a pore pressure (P) of 100 MPa, such conditions being met in the deeper parts of some basins.

2. Description of the Prior Art

A major challenge in petroleum and gas exploration is to delay to the maximum the hydrocarbon resources depletion. In such a context, zones that have hardly been explored due to the drilling costs linked with high temperature (T) and pressure (P) conditions now start to be considered as potentially economic. However, the hydrocarbons encountered there, predominantly methane (CH₄) of “thermogenic” origin (organic matter cracking), are often mixed with non-hydrocarbon gases, typically CO₂ and/or N₂, in such proportions that the reservoirs appear to be commercially unexploitable.

Thus, being able to assess, prior to drilling a zone of a sedimentary basin, whether the composition of the fluids accumulated in the traps may have been strongly influenced by the presence of non-hydrocarbon fluids is of considerable interest in exploration.

An approach aimed at predicting the level of a volatile constituent depending on its position in the sedimentary basin, from mineral reactions, has been presented specifically for CO₂ (L. M. Cathles & M. Schoell, 2007, “Modeling CO₂ Generation, Migration and Titration in Sedimentary Basins”, Geofluids Vol. 7, pp. 441-450). It calculates the CO₂ partial pressure, pCO₂ (or the fugacity thereof, fCO₂), from one or more “mineral buffers”, that is one or more assemblages of minerals considered to be present in the basin, and in a state of equilibrium. In doing so, it is not possible to assess the behavior of a particular lithology. On the contrary, the approach is a global one with the basin being taken as a whole. Any new basin study requires being able to estimate or calibrate the “capacity” of each mineral buffer selected, that is the average quantitative importance thereof in CO₂-producing zones of the basin being concerned.

### SUMMARY OF THE INVENTION

The method according to the invention allows calculation step by step of the balance of all the mineral reactions likely to produce volatile constituents during the geological history of a basin, with the only constraint of knowing how to assign compositions to the rocks considered as sources and of having access to the (T,P) path as a function of time.

Thus, the invention relates to a method of predicting an amount and a composition of fluids of mineral origin generated within a sedimentary basin by reactions occurring among sedimentary rock minerals as the rocks are buried during the geological history of the basin.

The invention thus provides a software tool for assessing the amount of essentially non-hydrocarbon fluids (H₂O, CO₂, N₂, etc.) generated in the basin, and the proportion of these fluids that may have migrated to reservoir rocks and mixed with hydrocarbons of organic origin, notably gaseous ones.

It represents a very significant advance to geologists looking for new fossil energy sources, in particular in deep reservoir rocks that may contain plentiful natural gas resources.

The invention relates to a method of predicting an amount and a composition of fluids of mineral origin generated within a sedimentary basin by reactions occurring among sedimentary rock minerals as the rocks are buried during the geological history of the basin, comprising:
i. acquiring geological data characteristic of the basin;
ii. constructing a representation of the basin by a grid;
iii. calculating, for at least one set of cells of the grid, using a basin model and of the geological data, the evolution of the parameters of a depth of burial (z), a temperature (T), a pore pressure (P), a volume (V) and a porosity φ at successive ages t, representative of the geological history of the basin;
iv. determining, in each cell of the set of cells, a mineralogical or chemical rock composition from the geological data of the basin,
v. determining an amount and composition of fluids of mineral origin within the set of cells using a geochemical model and from the parameters, the composition and a thermodynamic database.

According to the invention, after stage v, it is possible to determine an amount of the fluids that have migrated to reservoir rocks by determining thermodynamic properties of the fluids, such as the distribution in various fluid or solid phases and/or the densities and viscosities of the phases, by use of an equation of state and of a migration model dependent on the fluid properties.

The set of cells can correspond to source cells wherein a source cell is a cell wherein the rock composition, the pressure and the temperature favor the formation of fluids of mineral origin.

The temperature can be above 250° C. and the pressure is above 100 MPa.

In stage v, the following stages can be carried out:
determining a sequence of reactions wherein, along a given Temperature-Pressure path, a rock of given chemical composition goes through a succession of stable mineral compositions; and
determining variations in the amounts of minerals and in the amounts of fluid constituents exchanged during the sequence of reactions.

Variations in the amounts of minerals and of fluid constituents exchanged during the sequence of reactions can be determined by carrying out the following stages:
a. identifying a stable system for the age from the composition at an age tᵢ′
b. identifying mineral reactions causing change from the stable system for the age t, to the stable system for the age tᵢ₊₁;
c. carrying out a calculation of the quantitative balance, by mass and/or in number of moles, of the exchanges operated by these reactions;
d. carrying out a calculation of the quantitative balance, by volume, of these exchanges:
   i. from a thermodynamic database for the minerals,
   ii. from an equation of state allowing the composition and the density of each phase of the fluid to be calculated′
e. comparing volume variations obtained by geochemical modelling ᵢ₊₁ and by basin modelling Δᵢ₊₁ respectively: if δᵢ₊₁ exceeds Δᵢ₊₁ by an amount considered tolerable with regard to the expected precision of the fluid balances in the basin model, such as 1% in relative value for example, the composition of the system is modified by removing a volume δᵢ₊₁−Δᵢ₊₁ of fluid, either according to the composition of the total fluid, or that of the least dense phase, or that of a mixture of each phase in proportions determined according to the values taken by a property calculated for the fluid, such as viscosity;
f. storing the amount and the composition of the fluid subtracted from the system to set the new composition it takes at the age tᵢ₊₁; and
g. the new composition of the system becomes the composition taken into account for geochemical modelling upon passage to the next age (tᵢ₊₁→tᵢ₊₂).

The volume variation obtained by geochemical modelling can be written as follows:

δᵢ₊₁={(Vₘ)ᵢ₊₁+(V_{f})ᵢ₊₁}−{(Vₘ)+(V_{f})ᵢ},

where (V_{f})ᵢ and (Vₘ)ᵢ respectively represent the volume of fluid and the volume of minerals for the age tᵢ,
and the volume variation obtained by basin modelling is written as follows:

_{Δi+1}=Vᵢ₊₁−Vᵢ,

where Vᵢ represents the cell volume obtained by modelling.

The fluids can be characterized by high water and/or carbon dioxide and/or hydrocarbon gas and/or hydrogen and/or nitrogen and/or hydrogen sulfide contents.

### BRIEF DESCRIPTION OF THE DRAWINGS

FIG. 1 illustrates the operating sequence for implementing the method according to the invention;

FIG. 2A is a vertical section in the basin model taken as an example with a location of a source cell (MS) and of a reservoir level (NR), and T-P-z-φ is a path reconstructed by the model for the source cell; and FIG. 2B illustrates the evolution of porosity and depth of the source cell highlighted in the top portion of FIG. 2A with the evolution being according to the Age/Temperature/Pressure of the studied basin, a given age corresponding to a given Pressure (P) and a given Temperature (T);

FIG. 3 illustrates the evolution of composition parameters calculated by geochemical model taken along the T-P progression with FIG. 3A illustrating the mineral phases and water and carbon dioxide in the fluid, expressed in mol quantities, FIG. 3B illustrating, an aqueous phase (H₂O) and a vapor phase (CO₂), expressed in vol %, FIG. 3C illustrating in detail the composition for an aqueous phase (H₂O) and a vapor phase (CO₂) plus H₂O present in solids (mineral phases), expressed in mol quantities, and FIG. 3D illustrating relative variations of minerals, fluid and bulk volumes, expressed in vol %;

FIG. 4 shows a comparison between fluid composition calculated by the geochemical model, wherein FIG. 4A illustrates when the system is considered as closed along the whole T-P evolution, and FIG. 4B illustrates when part of the fluid is extracted from the system at ages of 25 Ma and 22 Ma, and the evolution which is calculated for ages older than 25 Ma in FIG. 2B is the same as in FIG. 4A and the composition parameters are the same as in FIG. 3C.

FIG. 5A and FIG. 5B respectively are maps of the cumulative amounts of H₂O and CO₂ expelled at the age of 22 Ma, and FIGS. 5C and 5D respectively are maps of the cumulative amounts of H₂O and CO₂ expelled at the age of 25 Ma; and

FIG. 6A and FIG. 6B respectively are block diagrams at the age of 22 Ma and at the age of 25 Ma. DETAILED DESCRIPTION OF THE INVENTION

Quantitative prediction of non-hydrocarbon fluids generated in basins is possible provided that a series of technical issues can be solved.

First, the reactions likely to deliver such fluids have to be identified for a given rock composition, as well as the T and P conditions under which they occur, and the balance thereof.

The mineral constituents of sedimentary rocks are derived from a sequence of processes:
continental weathering, which produces in particular large amounts of more or less hydrated clay minerals,
transport to depositional areas, together with grain size distribution and mixing, notably with carbonate minerals from various marine organisms,
modifications following deposition, referred to as “diagenetic”, allowing new minerals to precipitate within the pores, or in place of other minerals that dissolve.

The rather wide spectrum of mineral compositions observed, for example in the cores collected while drilling, results from all these often long and complex processes. As long as the depth of burial (z) of the sediments is moderate, which is translated into still modest temperature (T) and pressure (P) values (typically, T below 100° C. or 150° C., P below 20 or 30 MPa), the detrital minerals whose composition is rich in water (typically clays, for example smectites with two interfoliar water layers) remain stable. Then, in an increasingly marked manner as parameters z, T and P increase, the mineral assemblages tend to turn into new assemblages poorer in H₂O (or into OH groups). This also applies to other groups of atoms present in the crystal structure of the minerals, which can turn into volatile constituents (such as CO₂ or N₂) insofar as reactions can cause change from one mineral assemblage to another, more stable one.

The reactions mentioned are geochemical processes known in Earth sciences to describe “devolatilization” in the sphere of “metamorphism” (P. Eskola, 1921, “The Mineral Facies of Rocks”, Norsk Geologisk Tidsskrift Vol. 6, pp. 142-194; N. L. Bowen, 1940, “Prograde Metamorphism of Siliceous Limestone and Dolomite, Journal of Geology Vol. 48, pp. 225-274).

In some basins where the geothermal gradient and/or the thickness of the deposits are great, the temperature and pressure conditions reached in the more deeply buried sediments are such (above 400° C. and 200 MPa) that large amounts of volatile constituents can be formed by these reactions. The nature of the mineral assemblages involved, the reactions causing them to change from one to the next and the conditions under which these reactions occur can be predicted from:

(1) a sufficiently detailed description of the thermodynamic properties of the existing minerals, accessible today in dedicated databases (some of which have been published),

(2) a computer code allowing, from these thermodynamic properties and a rock composition, to calculate which assemblage is the most stable under all conditions and which amounts of matter are exchanged in the successive reactions driving the system towards the most stable state. This type of code is referred to as “geochemical modelling tool”. There are many of them, intended for studying metamorphism.

The second issue to be settled relates to the volume variations induced by the reactions. The molecules released by the above mineral evolutions (H₂O, CO₂, N₂, etc.) are in the fluid state under the conditions being examined. The reactions modify the mineral composition, that of the fluid, and therefore the volumes of mineral matter and of fluid. Very generally, the volume of the fluid increases. Since the volume variation of the rock (minerals+fluid) is constrained by the evolution of the basin geometry and of the “compaction” (or settling) of the sediments, two parameters described by a standard basin modelling tool such as the TemisFlow® software (IFP Energies nouvelles, France), it is possible to calculate simply, depending on the reaction progress and under the assumption that the influence of the reactions on the pressure is not taken into account, the proportion of fluid that can remain in place in the pore volume, and the proportion that could be expelled.

An additional issue concerns the nature and the behavior of the fluid. Depending on the composition thereof and on the T and P values, the fluid mixture has one or more phases. In the calculation of the expelled proportion of fluid as presented above, it may be desirable to deal in a different manner with each phase, according to the value taken by some of the respective characteristics thereof (density, viscosity). The nature and the behavior of the fluid can be predicted by the conjunction (1) of an “equation of state” (often a cubic equation relating pressure, volume and temperature, and integrated in a computer code), capable of calculating the state of the phases of the fluid, their composition and their volume, therefore their density; (2) of parameterized models capable of calculating other possibly required quantities such as the viscosity for example.

The last issue relates to the migration of the expelled fluid. Within the context of the method that is the object of this presentation, the migration is considered by means of a ray-tracing procedure already included in basin modelling.

The method according to the invention combines, in a sequence of coherent stages, the solutions that can be provided for each of these technical issues.

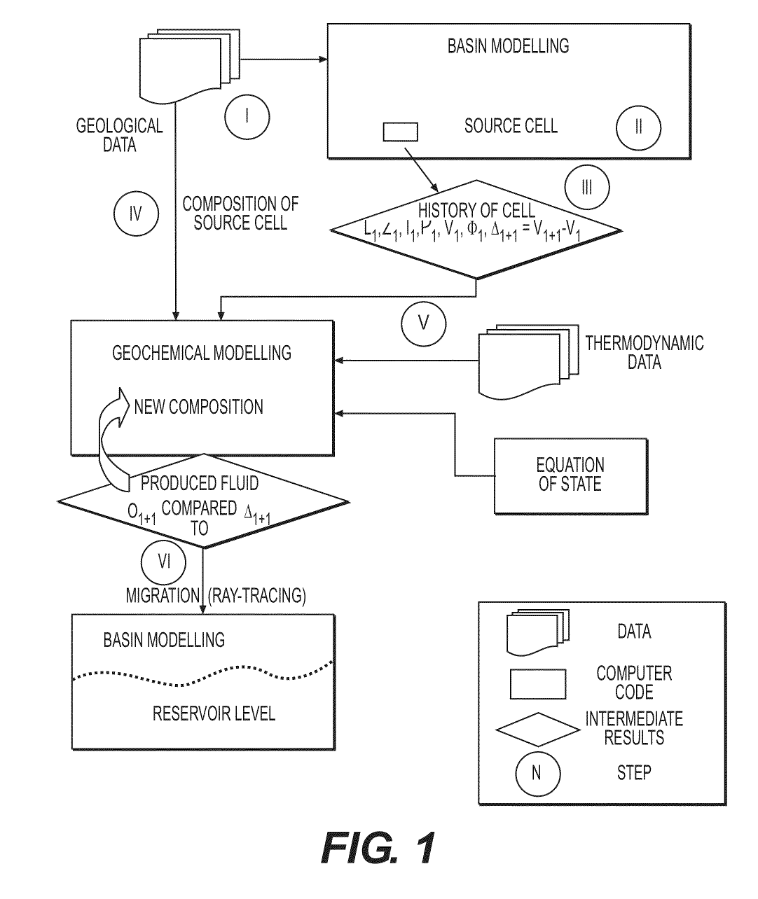

FIG. 1 illustrates the stages of the method according to the invention for predicting the amount and the composition of fluids of mineral origin generated in a sedimentary basin. This method comprises the following stages:
i. Acquisition of geological data characteristic of the sedimentary basin
ii. Construction of a representation of the basin by a grid
iii. Calculation of physical parameters (z, T, P, V, φ) for various ages
iv. Determination of the mineralogical or chemical composition of the source rocks
v. Determination of the amount and composition of the fluids of mineral origin in the source rocks
   and an optional stage:
vi. Migration of the fluids of mineral origin in the reservoirs.

According to the invention, a fluid of mineral origin is characterized by high water and/or carbon dioxide and/or hydrocarbon gas and/or hydrogen and/or nitrogen and/or hydrogen sulfide contents.

i. Acquisition of Geological Data Characteristic of the Sedimentary Basin

The geological data required for basin modelling are partly described in the following document:
J. L. Rudkiewicz et al., 2000, “Integrated Basin Modeling Helps to Decipher Petroleum Systems”; R. Melo and B. J. Katz, eds, “Petroleum Systems of South Atlantic Margin”, AAPG Memoir, Vol. 73, pp. 27-40.).

The data involved are at least:
geometry of the layers at the current time in one (z), two (x,z) or three (x,y,z) dimensions,
nature of the rocks making up the layers,
geological age of the rocks,
temperatures, pressures, porosities observed in boreholes, and
if necessary, eroded sediment thicknesses and erosion ages.

These data are acquired by known techniques (logging, coring, etc.).

ii. Construction of a Representation of the Basin by a Grid

Constructing a representation of a sedimentary basin is a well-known stage. The grid can have one (z), two (x,z) or three (x,y,z) dimensions.

iii. Calculation of Physical Parameters (z, T, P, V, φ) for Various Ages

In this stage, the evolution of the following parameters is determined for a set of cells of the grid: a depth of burial (z), a temperature (T), a pore pressure (P), a volume (V) and a porosity (φ) at successive ages (tᵢ) representative of the geological history of the basin.

A basin modelling software tool referred to as “basin model” and the geological data acquired in stage i are used. A basin model is a software dedicated for sedimentary basin modelling, capable of calculating, in any cell and for any geological time (t), the values reached by depth of burial (z), temperature (T), pore pressure (P), volume (V) and porosity φ. Note: V(z) and φ(z) define what is referred to as “compaction”. TemisFlow® (IFP Energies nouvelles, France) is an example of a basin model.

“Grid cell records”, that is charts {tₕ zᵢ, Tᵢ, Pᵢ, Vᵢ, φᵢ} containing the value of z, T, P, V and φ of a cell at successive times t, representative of the geological history of the basin (sedimentary layer deposition or erosion episodes), are thus determined from this software used on a computer.

The reconstruction of these records can be limited to the cells that are considered to be potential sources of fluids of mineral origin. These cells are referred to as source cells. A source cell can be defined as a cell wherein the rock composition, the pressure and the temperature favor the formation of fluids of mineral origin, the pressure and the temperature having reached a given threshold during their geological evolution (for example a temperature above 250° C. and a pressure above 100 MPa).

iv. Determination of the Mineralogical or Chemical Composition of the Source Rocks

In this stage, the representative mineralogical (or chemical) composition is determined for each source cell, that is a set of minerals likely to describe in a manner considered to be sufficiently representative and complete the composition of the rocks of the basin in this cell.

This mineralogical (or chemical) composition is inferred from the geological data of the basin acquired in the initial stage. It can be determined directly by mineralogical analysis of the cuttings returned to the surface while drilling one or more holes, or by mineralogical examination of rock cores taken during such drilling operations. It can also be determined indirectly through analysis of the logs recorded along the wells. Another method uses the indirect geophysical measurements (seismic reflection, magnetic survey or borehole gravity) that provide information on the main composition classes. Finally, a last method seeks lithologic assemblies that come as lateral equivalents of the layers of interest and that outcrop nearby.

v. Determination of the Amount and Composition of the Fluids of Mineral Origin in the Source Rocks

In this stage, the amount and the composition of the fluids of mineral origin are determined within the set of source cells using:
a geochemical model,
parameters z, T, P, V and φ,
the mineralogical or chemical composition of the source rocks,
a thermodynamic database containing, for a set of minerals likely to describe the composition of the rocks of the basin, the thermodynamic parameters allowing knowing and/or calculating under all temperature and pressure conditions the Gibbs free energy of each mineral and the molar volume of each mineral.

A geochemical model (Theriak/Domino for example) is a software capable of calculating under all temperature and pressure conditions, and from the thermodynamic database:

the stable mineral composition of a rock having a given chemical composition,

the sequence of reactions causing the rock to go, along a given T-P path, through a succession of stable compositions,

the amounts of minerals and of fluid constituents exchanged during this sequence of reactions.

This stage v thus comprises the following substages:

determining a sequence of reactions causing the rock to go, along a given Temperature-Pressure path, through a succession of stable mineral compositions,
determining variations in the amounts of minerals and in the amounts of fluid constituents exchanged during the sequence of reactions.

The following iterations are therefore performed for each source cell:
a. identifying a stable system (mineral assemblage+fluid) for the age tᵢ₊₁ (conditions Tᵢ₊₁, Pᵢ₊₁) from the composition at an age tᵢ,
b. identifying mineral reactions causing change from the stable system for the age tᵢ to the stable system for the age tᵢ₊₁,
c. carrying out a calculation of the quantitative balance, by mass and/or in number of moles, of the exchanges operated by these reactions (that is minerals created or destructed, fluid constituents created or destructed),
d. carrying out a calculation of the quantitative balance, by volume, of these exchanges:
   from a thermodynamic database for the minerals,
   from an equation of state allowing the composition and the density of each phase of the fluid to be calculated; an example of equation of state is given in the following document:
   Z. Duan, N. Moller & J. H. Weare, 1992, “An Equation of State (EOS) for CH₄CO₂H₂O system: I. Pure Systems from 0 to 1000° C. and 0 to 8000 Bar”, Geochimica Cosmochimica Acta, Vol. 56, pp. 2605-2617,
e. comparing volume variations obtained by geochemical modelling δᵢ₊₁ and by basin modelling (compaction modelling) Δᵢ₊₁ respectively: if δᵢ₊₁ exceeds Δᵢ₊₁ by an amount considered tolerable with regard to the expected precision of the fluid balances in the basin model, such as 1% in relative value for example, the composition of the system is modified by removing a volume δᵢ₊₁−Δᵢ₊₁ of fluid, either according to the composition of the total fluid, or that of the least dense phase, or that of a mixture of each phase in proportions determined according to the values taken by a property calculated for the fluid, such as viscosity,
f. storing the amount and the composition of the fluid subtracted from the system to set the new composition it takes at the age tᵢ₊₁,
g. the new composition of the system becomes the composition taken into account for geochemical modelling upon passage to the next age (tᵢ₊₁→tᵢ₊₂).

The volume variation obtained by geochemical modelling can be written, in the source cell:

δᵢ₊₁={(Vₘ)ᵢ₊₁+(V_{f})ᵢ₊₁}−{(Vₘ)ᵢ+(V_{f})ᵢ},

where (V_{f})ᵢ and (Vₘ)ᵢ respectively represent the volume of fluid and the volume of minerals for the age tᵢ.

The volume variation obtained by basin modelling is written as follows:

_{Δi+1}+Vᵢ₊₁−Vᵢ,

where Vᵢ represents the volume of the source cell obtained by basin modelling.
vi. Migration of the Fluids of Mineral Origin in the Reservoirs

The amount of fluids that have migrated to reservoir rocks is determined. The properties of the fluid (phase state, composition and density of each phase) are therefore determined by an equation of state that is usable as soon as the temperature, the pressure and the overall composition of the fluid are known.

Finally, these properties are used within a migration model. A ray-tracing procedure is one possible option for migration calculation. This type of procedure is available in the TemisFlow® basin modelling tool (IFP Energies nouvelles, France) for example.

This procedure is applied to the age by considering that the fluid masses subtracted from the source cells, obtained for the time iteration t, during operation (v) described above (Determination of the amount and composition of the fluids of mineral origin in the source rocks), are collected instantly in one (or optionally more) “reservoir level(s)” previously defined in the geological data.

When appropriate, they join there hydrocarbon fluids from the mother rocks conventionally taken into account by a basin model. The geometry of the given reservoir level at the age determines the existence therein of fluid “traps”.

Through a new use of the equation of state, the composition and the density of the fluid phase(s) are calculated under the temperature (Tᵢ₊₁) and pressure (Pᵢ₊₁) conditions of each trap at the age tᵢ₊₁.

The fluid distribution in the traps occurs according to these properties and to the volume available in each trap. If several reservoir levels are defined, a distribution rule is also defined for the deep fluids collected between the various levels.

### EXAMPLE

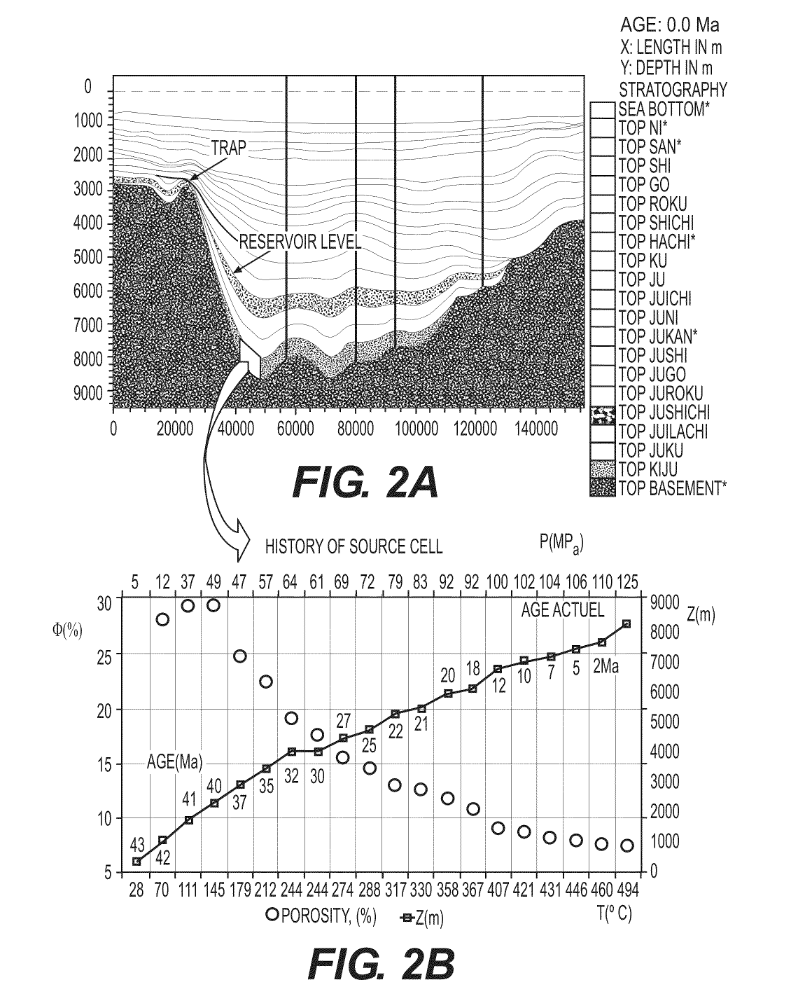

FIGS. 2 to 6 illustrate an application example for the method according to the invention. The geological characteristics according to this example are as follows:

Parameters Related to the Sedimentary Basin

FIGS. 2A and 2B respectively show a geologic section through the basin representation selected (3D grid) and the history of a source cell located in the deepest layer (deposited between 43 and 42 Ma).

For the source cell, the basin model is used to calculate the evolution of the depth (top of the cell), of the temperature, of the pressure and of the porosity throughout the 19 stages of the geological history, that is in the intervals contained between the 20 ages of the temporal description of the basin (43, 42, 41, 40, 37, 35, 32, 30, 27, 25, 22, 21, 20, 18, 12, 10, 7, 5 and 2 Ma before 0 Ma), and of the volume of the cell (not shown).

Mineralogical Composition of the Source Cell:

sandstone containing diagenetic carbonates, expressed with the chemical elements Si, Al, Ca, Mg, C, H and O (neither Na nor K or Fe):
proportions in volume, defined at 100° C. and 32.5 MPa: 60% quartz, 5% kaolinite, 15% calcite, 10% dolomite, porosity (I) 10% occupied by a fluid consisting of 90% H₂O and 10% CO₂;
corresponding elemental composition, in number of moles for 1 dm³ rock (rock=minerals+fluid present in the pores): 27.451 mol Si (silicon), 1.005 mol Al (aluminium), 5.615 mol Ca (calcium), 1.554 mol Mg (magnesium), 7.724 mol C (carbon), 12 mol H (hydrogen), 85.026 mol O (oxygen).
Fluid collection reservoir level for the ray-tracing procedure: layer deposited between 40 and 37 Ma (FIG. 2).

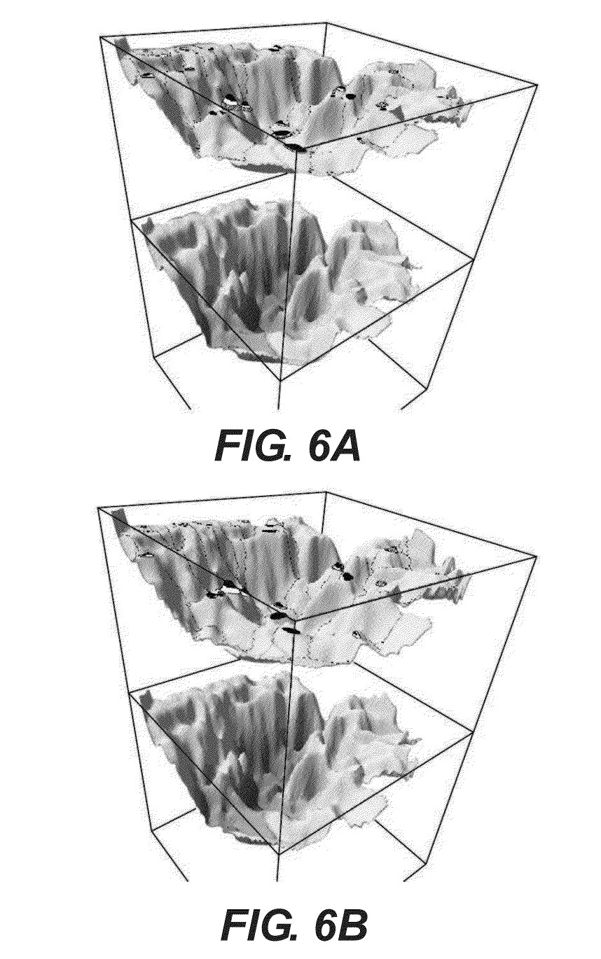

Basin structure: it is illustrated by the block diagram of FIG. 6.

The software tools used in this example are:

1. Basin modelling software (basin model): TemisFlow® (IFP Energies nouvelles, France). Other basin models could be used to the same end.

2. Thermodynamic database: it is constructed on the basis of data from the following references:
R. G. Berman: 1988, “Internally-Consistent Thermodynamic Data for Minerals in the System Na₂O—K₂O—CaO—MgO—FeO—Fe₂O₃—Al₂O₃—SiO₂—TiO₂—H₂O—OO₂”, Journal of Petrology, Vol. 29, pp. 485-522,
O. Vidal, T. Parra & F. Trotet, 2001, “A Thermodynamic Model for Fe—Mg Aluminous Chlorite Using Date from Phase Equilibrium Experiments and Natural Pelitic Assemblages in the 100°-600° C., 1-25 kb range”, American Journal of Science, Vol. 301, pp. 557-592,
T. Parra, O. Vidal & P. Agard, 2002, “A Thermodynamic Model for Fe—Mg Dioctahedral K-White Micas Using Data from Phase Equilibrium Experiments and Natural Pelitic Assemblages”, Contribution to Mineralogy and Petrology, Vol. 143, pp. 706-732,
O. Vidal, T. Parra & P. Vieillard, 2005, “Thermodynamic Properties of the Tschermak Solid Solution in Fe-chlorites: Application to Natural Examples and Possible Role of Oxidation”, American Mineralogist, Vol. 90, pp. 359-370,
O. Vidal, V. De Andrade, E. Lewin, M. Munoz, T. Parra & S. Pascarelli, 2006, “P-T-Deformation-Fe³⁺/Fe²⁺ Mapping at the Thin Section Scale and Comparison with XANES Mapping. Application to a Garnet-Bearing Metapelite from the Sambagawa Metamorphic Belt (Japan)”, Journal of Metamorphic Geology, Vol. 24, pp. 669-683).

Other databases could be used to the same end, for example that of T. J. B. Holland & R. Powell (2011, “An Improved and Extended Internally Consistent Thermodynamic Dataset for Phases of Petrological Interest, Involving a New Equation of State for Solids”, Journal of Metamorphic Geology, Vol. 29, pp. 333-383).

3. Computer code for geochemical modelling (geochemical model): the Theriak/Domino suite has been used. This suite is described in the following references:
C. de Capitani & T. H. Brown, 1987, “The Computation of Chemical Equilibrium in Complex systems Containing Non-Ideal Solutions”, Geochimica Cosmochimica Acta, Vol. 51, pp. 2639-2652,
C. de Capitani & K. Petrakakis K., 2010, “The Computation of Equilibrium Assemblage Diagrams with Theriak/Domino Software”, American Mineralogist, Vol. 95, pp. 1006-1016).

Other computer codes could be used to the same end, for example Perple_X (J. A. D. Connolly, 2005, “Computation of Phase Equilibria by Linear Programming: A Tool for Geodynamic Modeling and its Application to Subduction Zone Decarbonation”, Earth & Planetary Science Letters, Vol. 236, pp. 524-541), or THERMOCALC (R. Powell, T. J. B. Holland & B. Worley, 1998, “Calculating Phase Diagrams Involving Solid Solutions via Non-Linear Equations, with Examples Using THERMOCALC”, Journal of Metamorphic Geology, Vol. 16, pp. 577-588).

4. Equation of state by Z. Duan, N. Moller & J. H. Weare, described in:
1992, “An Equation of State (EOS) for CH₄CO₂H₂O System: I. Pure Systems from 0 to 1000° C. and 0 to 8000 Bar”, Geochimica Cosmochimica Acta, Vol. 56, pp. 2605-2617,
1992, “An Equation of State (EOS) for CH₄CO₂H₂O System: II. Mixtures from 50 to 1000° C. and 0 to 1000 Bar”, Geochimica Cosmochimica Acta, Vol. 56, pp. 2619-2631).

Other equations of state could be used to the same end.

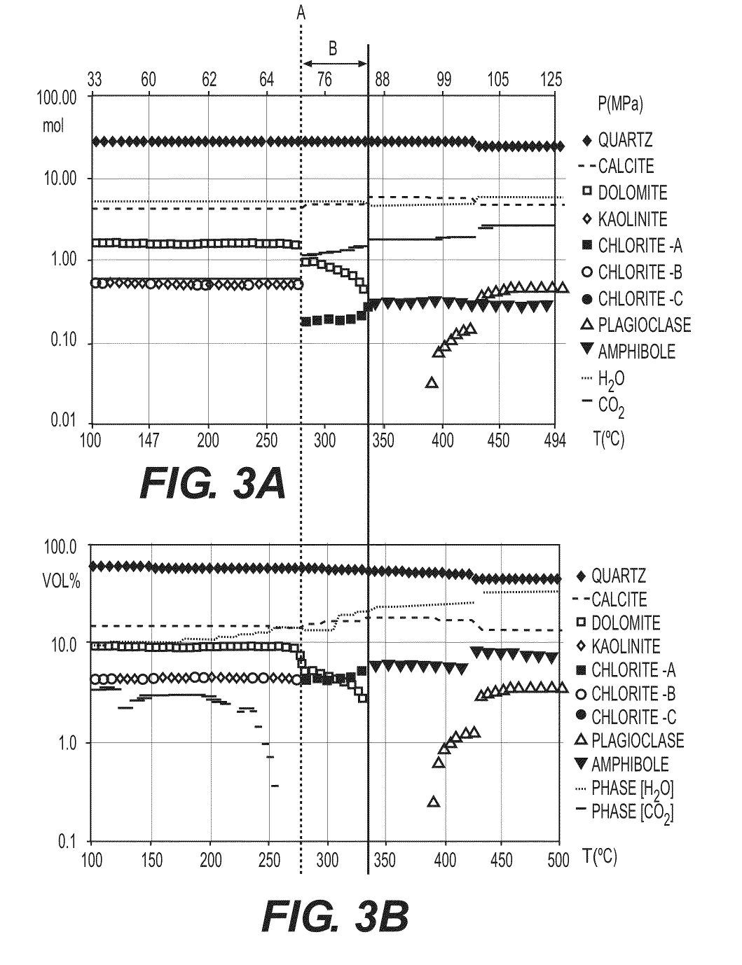

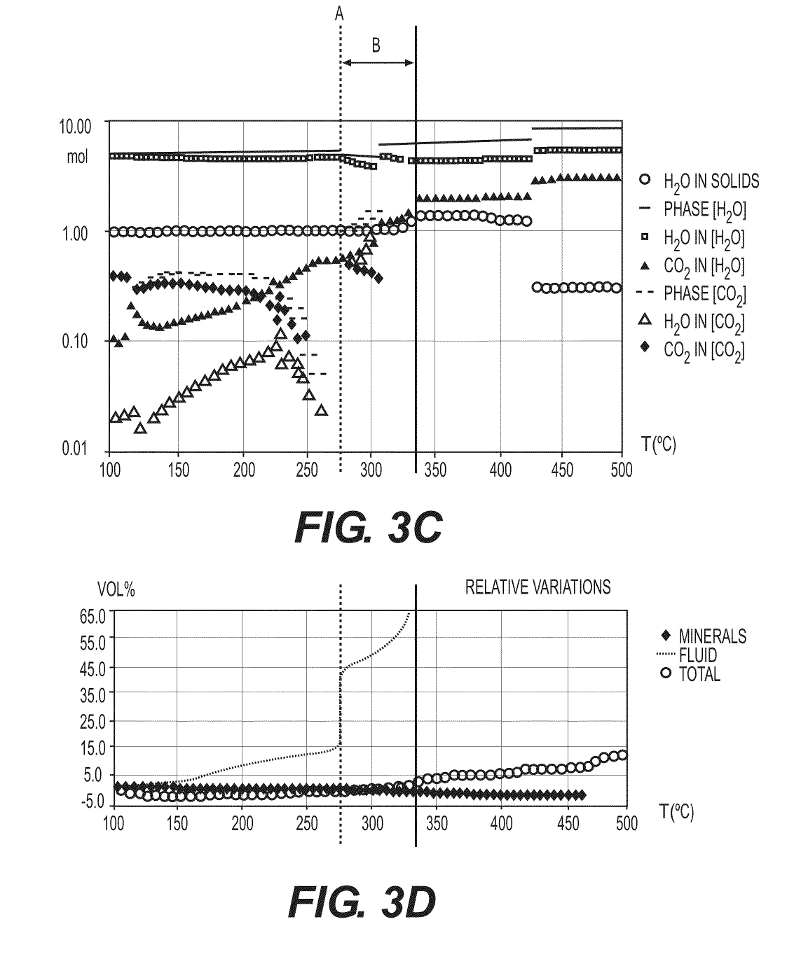

Closed-system geochemical modelling, that is without fluid subtraction, contributes to a better understanding of a standard evolution of the compositions and the volumes along the “{tᵢ, Tᵢ, Pᵢ} path” (path travelled at increasing T and P), for the chemical system representing source cell No. 1 (FIG. 3):
standard event A: expresses the effect of a fluid-producing univariant reaction leading to a sudden composition and volume variation (reaction A: dolomite+kaolinite+H₂O→[chlorites]+quartz+calcite+CO₂ complete until kaolinite depletion) (the notation [chlorites] represents the chlorites “solid solution”),
standard episode B: expresses the effect of a divariant reaction where the composition of the chlorites solid solution gradually changes (dolomite+quartz+[chlorites 1]+H₂O→[chlorites 2]+calcite+CO₂ until dolomite depletion),
variation of the total volume {(Vₘ)ᵢ+(V_{f})} (FIG. 3D): low and negligible before event A; positive during event A; positive and progressive during episode B.

Event A occurs between the times “27 Ma” and “25 Ma” of the basin modelling. It induces the exit of a fluid “parcel” at the age “25 Ma”. Episode B progressively takes place between the ages “25 Ma” and “22 Ma”. It continues a little beyond that time, but from the age “22 Ma” the fluid it produces can be taken into account for the migration. The ages “25 Ma” and “22 Ma” allow to illustrate here the procedure of opening of the source system and of modification in the composition of the source grid related thereto, then that of the fluid migration to a reservoir level overlying the source.

The implementation of the method can thus be illustrated as follows by using this example:

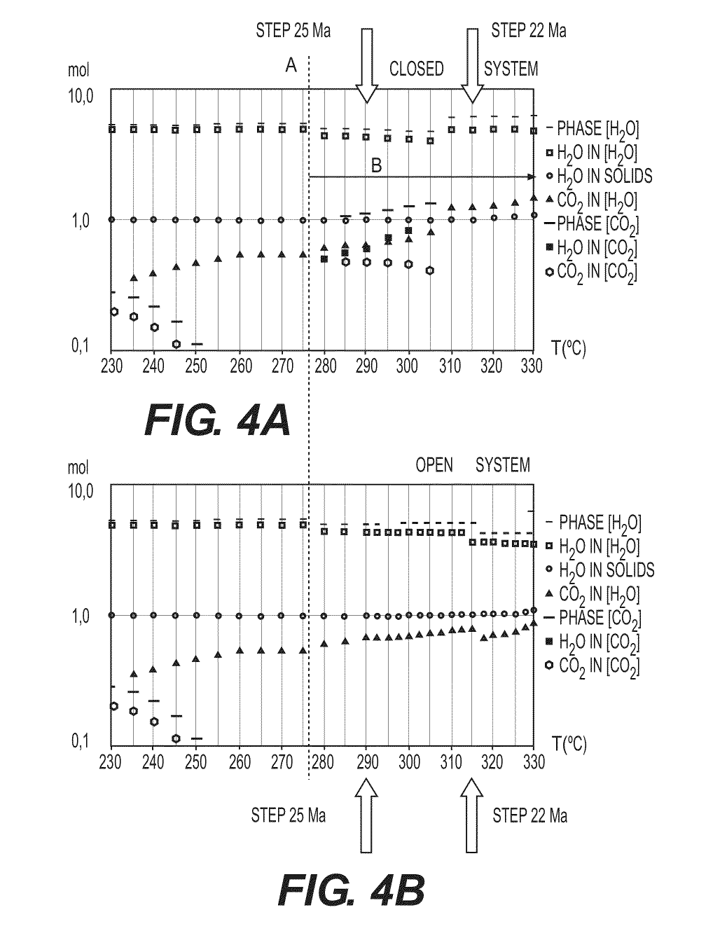

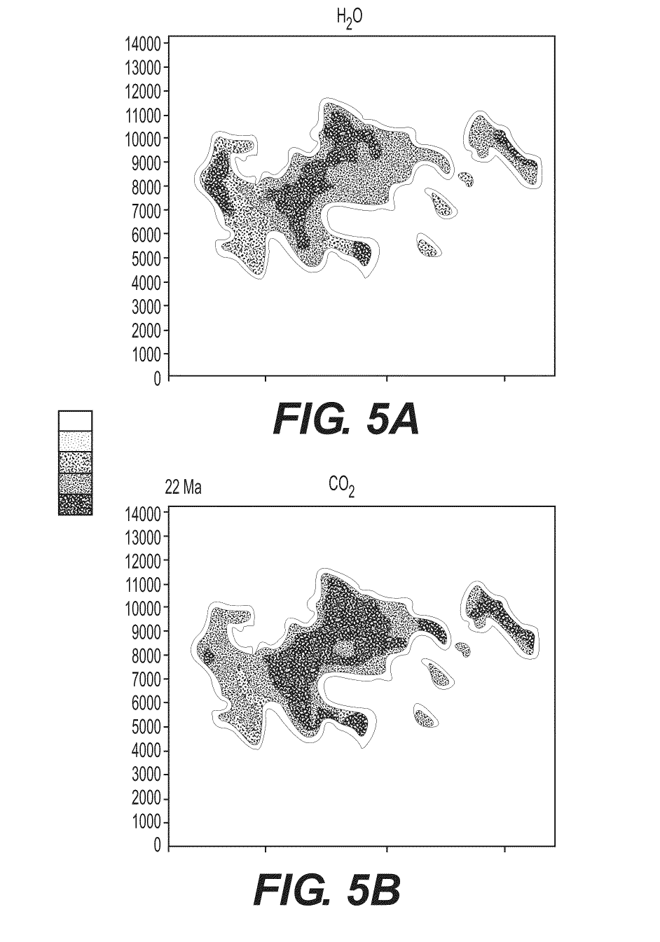

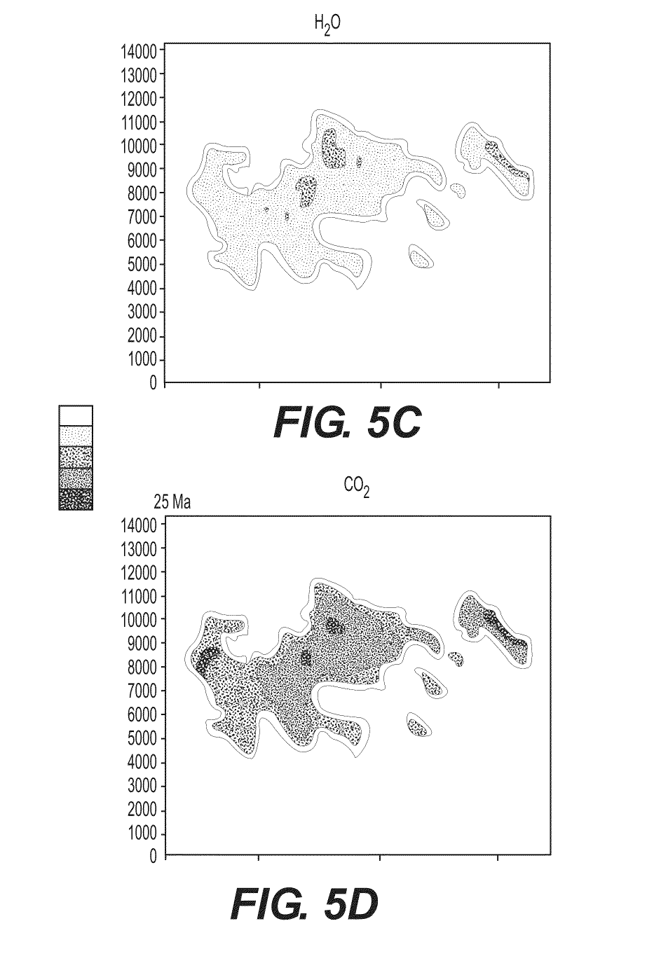

1. Basin modelling: reconstruction of the {tᵢ, zᵢ, Tᵢ, Pᵢ, Vᵢ, φᵢ} record of a source cell (FIG. 2).
2. Geochemical modelling and use of the equation of state, step by step, for each source cell. For example, between the times “43 Ma” and “25 Ma”, for the chemical system representing the source cell:
a. Case where, at each age tᵢ₊₁, the variation of the total volume δᵢ₊₁−Δᵢ₊₁ remains within the tolerance defined for the source system to remain closed (as in FIG. 3 before event A): The composition of the system is not modified for the geochemical calculation in the next stage,
b. Case where the variation of the total volume requires extraction of part of the fluid from the source system, as at the age “25 Ma” (FIG. 3, after event A) and at the age “22 Ma” (FIG. 3, during episode B):
   i. the equation of state gives the composition of each fluid phase at the age “25 Ma” (FIG. 4A),
   ii. a separation rule is applied: here, it is decided to remove the fluid phases one after the other, in order of increasing density, which leads to remove all of the volume of the “CO₂ phase” formed (i.e. approximately 5% of the total volume of the rock), or H₂O and CO₂ in molar proportion of their contribution to this phase (56% and 44% respectively),
   iii. the composition of the system is modified for the geochemical calculation between the age “25 Ma” and the age “22 Ma” (FIG. 4B): no CO₂ phase re-forms, but at 22 Ma it is advisable to remove 17% of the “H₂O phase” containing 85% H₂O and 15% CO₂ respectively in molar proportion (the procedure would be continued with a new change in composition to resume the geochemical calculation, up to the age “21 Ma”, which is not illustrated here).
      3. Basin modelling: for the migration calculation using the ray-tracing procedure. At a given stage, the procedure is to collect the fluids from a set of source cells (inorganic fluids and possibly hydrocarbons from otherwise identified mother rocks). In a single reservoir level here.
a. The first stage where the migration is illustrated ends at “25 Ma”: a set of source cells is concerned, for which the amounts of H₂O and CO₂ to be expelled are shown (FIG. 5, bottom),
b. The migration through ray-tracing, at 25 Ma, of H₂O and CO₂ produced in the source cells is combined with that of CH₄ produced in mother rocks: Application of the equation of state within each structural trap determined by the basin geometry at 25 Ma leads to the fluid distribution shown in FIG. 6A. A CO₂-rich phase, the lightest one, occupying the upper part of the traps, and a “hydrocarbon” phase (also produced in mother rocks), relatively denser, can be seen. Water forms the third phase and it occupies the base of the pore space available in the structure of the trap, within the reservoir rock,
c. The second stage where the migration is illustrated ends at “22 Ma”: FIG. 5A (top) shows, for this age, the cumulative amounts of H₂O and CO₂ expelled since 25 Ma,
d. Similarly, the migration through ray-tracing at 22 Ma leads to the fluid distribution shown in FIG. 6B. The fluids present at 25 Ma add up to those expelled between 25 and 22 Ma, and they provide a new phase distribution at 22 Ma. It can be noted that the traps available at 22 Ma are not exactly the same as those available at 25 Ma.

1-8. (canceled)

9. A method of predicting an amount and a composition of fluids of mineral origin generated within a sedimentary basin by reactions occurring among sedimentary rock minerals as the rocks are buried during the geological history of the basin, comprising:
i. acquiring geological data characteristic of the basin;
ii. constructing a representation of the basin by a grid;
iii. calculating, for at least one set of cells of the grid, by using a basin model of the geological data, an evolution of a depth of burial, a temperature, a pore pressure, a volume and a porosity at successive ages representative of the geological history of the basin;
iv. determining, in each cell of the set of cells, a mineralogical or chemical rock composition from the geological data of the basin; and
v. determining an amount and a composition of fluids of mineral origin within the set of cells using a geochemical model and from the parameters and the mineralogical or chemical rock composition, and a thermodynamic database.

10. A method as claimed in claim 9 wherein, after step v, determining an amount of the fluids that have migrated to reservoir rocks is determined by determining thermodynamic properties of the fluids, including a distribution in fluid or solid phases and/or densities and viscosities of the phases, using an equation of state and a migration model dependent on the fluid properties.

11. A method as claimed in claim 9, wherein the set of cells corresponds to source cells including a rock composition, a pressure and a temperature which favor formation of fluids of mineral origin.

12. A method as claimed in claim 10, wherein the set of cells corresponds to source cells including a rock composition, a pressure and a temperature which favor formation of fluids of mineral origin.

13. A method as claimed in claim 11, wherein the temperature is above 250° C. and the pressure is above 100 MPa.

14. A method as claimed in claim 12, wherein the temperature is above 250° C. and the pressure is above 100 MPa.

15. A method as claimed in claim 9 wherein, in step v, comprising:
determining a sequence of reactions wherein, along a given temperature-pressure path, a rock of given chemical composition goes through a succession of stable mineral compositions; and
determining variations in amounts of minerals and in amounts of fluid constituents exchanged during sequence of reactions.

16. A method as claimed in claim 10 wherein, in step v, comprising:
determining a sequence of reactions wherein, along a given temperature-pressure path, a rock of given chemical composition goes through a succession of stable mineral compositions; and
determining variations in amounts of minerals and in amounts of fluid constituents exchanged during sequence of reactions.

17. A method as claimed in claim 11 wherein, in step v, comprising:
determining a sequence of reactions wherein, along a given temperature-pressure path, a rock of given chemical composition goes through a succession of stable mineral compositions; and
determining variations in amounts of minerals and in amounts of fluid constituents exchanged during sequence of reactions.

18. A method as claimed in claim 12 wherein, in step v, comprising:
determining a sequence of reactions wherein, along a given temperature-pressure path, a rock of given chemical composition goes through a succession of stable mineral compositions; and
determining variations in amounts of minerals and in amounts of fluid constituents exchanged during sequence of reactions.

19. A method as claimed in claim 13 wherein, in step v, comprising:
determining a sequence of reactions wherein, along a given temperature-pressure path, a rock of given chemical composition goes through a succession of stable mineral compositions; and
determining variations in amounts of minerals and in amounts of fluid constituents exchanged during sequence of reactions.

20. A method as claimed in claim 14 wherein, in step v, comprising:
determining a sequence of reactions wherein, along a given temperature-pressure path, a rock of given chemical composition goes through a succession of stable mineral compositions; and
determining variations in amounts of minerals and in amounts of fluid constituents exchanged during sequence of reactions.

21. A method as claimed in claim 15, wherein variations in amounts of minerals and fluid constituents exchanged during the sequence of reactions are determined by a process comprising:
a. identifying a stable system for an age ti+1 from a composition at an age ti;
b. identifying mineral reactions causing change from a stable system for the age ti to a stable system for an age ti+1;
c. carrying out a calculation of quantitative balance, by mass and/or in number of moles, of the exchanges operated by the reactions;
d. carrying out a calculation of quantitative balance, by volume, of the exchanges by involving:
i. a thermodynamic database for the minerals,
ii. an equation of state allowing a composition and a density of each phase of the fluid to be calculated;
e. comparing volume variations obtained by geochemical modelling δi+1 and by basin modelling Δi+1 respectively if δi+1 exceeds Δi+1 by an amount determined with regard to an expected precision of fluid balances in a basin model, the composition of the system being modified by removing a volume δi+1−Δi+1 of fluid, either according to a composition of a total fluid, or of a least dense phase, or of a mixture of each phase in proportions determined according to values taken by a property calculated for the fluid, including viscosity;
f. storing an amount and composition of fluid subtracted from the system to set a composition acquired at the age ti+1; and
g. a new composition of the system accounted for geochemical modelling upon passage to the next age (ti+1→ti+2).

22. A method as claimed in claim 16, wherein variations in amounts of minerals and fluid constituents exchanged during the sequence of reactions are determined by a process comprising:
a. identifying a stable system for an age ti+1 from a composition at an age ti;
b. identifying mineral reactions causing change from a stable system for the age ti to a stable system for an age ti+1;
c. carrying out a calculation of quantitative balance, by mass and/or in number of moles, of the exchanges operated by the reactions;
d. carrying out a calculation of quantitative balance, by volume, of the exchanges by involving:
i. a thermodynamic database for the minerals,
ii. an equation of state allowing a composition and a density of each phase of the fluid to be calculated;
e. comparing volume variations obtained by geochemical modelling δi+1 and by basin modelling Δi+1 respectively if δi+1 exceeds Δi+1 by an amount determined with regard to an expected precision of fluid balances in a basin model, the composition of the system being modified by removing a volume δi+1−Δi+1 of fluid, either according to a composition of a total fluid, or of a least dense phase, or of a mixture of each phase in proportions determined according to values taken by a property calculated for the fluid, including viscosity;
f. storing an amount and composition of fluid subtracted from the system to set a composition acquired at the age ti+1; and
g. a new composition of the system accounted for geochemical modelling upon passage to the next age (ti+1→ti+2).

23. A method as claimed in claim 17, wherein variations in amounts of minerals and fluid constituents exchanged during the sequence of reactions are determined by a process comprising:
a. identifying a stable system for an age ti+1 from a composition at an age ti;
b. identifying mineral reactions causing change from a stable system for the age ti to a stable system for an age ti+1;
c. carrying out a calculation of quantitative balance, by mass and/or in number of moles, of the exchanges operated by the reactions;
d. carrying out a calculation of quantitative balance, by volume, of the exchanges by involving:
i. a thermodynamic database for the minerals,
ii. an equation of state allowing a composition and a density of each phase of the fluid to be calculated;
e. comparing volume variations obtained by geochemical modelling δi+1 and by basin modelling Δi+1 respectively if δi+1 exceeds Δi+1 by an amount determined with regard to an expected precision of fluid balances in a basin model, the composition of the system being modified by removing a volume δi+1−Δi+1 of fluid, either according to a composition of a total fluid, or of a least dense phase, or of a mixture of each phase in proportions determined according to values taken by a property calculated for the fluid, including viscosity;
f. storing an amount and composition of fluid subtracted from the system to set a composition acquired at the age ti+1; and
g. a new composition of the system accounted for geochemical modelling upon passage to the next age (ti+1→ti+2).

24. A method as claimed in claim 18, wherein variations in amounts of minerals and fluid constituents exchanged during the sequence of reactions are determined by a process comprising:
a. identifying a stable system for an age ti+1 from a composition at an age ti;
b. identifying mineral reactions causing change from a stable system for the age ti to a stable system for an age ti+1;
c. carrying out a calculation of quantitative balance, by mass and/or in number of moles, of the exchanges operated by the reactions;
d. carrying out a calculation of quantitative balance, by volume, of the exchanges by involving:
i. a thermodynamic database for the minerals,
ii. an equation of state allowing a composition and a density of each phase of the fluid to be calculated;
e. comparing volume variations obtained by geochemical modelling δi+1 and by basin modelling Δi+1 respectively if δi+1 exceeds Δi+1 by an amount determined with regard to an expected precision of fluid balances in a basin model, the composition of the system being modified by removing a volume δi+1−Δi+1 of fluid, either according to a composition of a total fluid, or of a least dense phase, or of a mixture of each phase in proportions determined according to values taken by a property calculated for the fluid, including viscosity;
f. storing an amount and composition of fluid subtracted from the system to set a composition acquired at the age ti+1; and
g. a new composition of the system accounted for geochemical modelling upon passage to the next age (ti+1→ti+2).

25. A method as claimed in claim 19, wherein variations in amounts of minerals and fluid constituents exchanged during the sequence of reactions are determined by a process comprising:
a. identifying a stable system for an age ti+1 from a composition at an age ti;
b. identifying mineral reactions causing change from a stable system for the age ti to a stable system for an age ti+1;
c. carrying out a calculation of quantitative balance, by mass and/or in number of moles, of the exchanges operated by the reactions;
d. carrying out a calculation of quantitative balance, by volume, of the exchanges by involving:
i. a thermodynamic database for the minerals,
ii. an equation of state allowing a composition and a density of each phase of the fluid to be calculated;
e. comparing volume variations obtained by geochemical modelling δi+1 and by basin modelling Δi+1 respectively if δi+1 exceeds Δi+1 by an amount determined with regard to an expected precision of fluid balances in a basin model, the composition of the system being modified by removing a volume δi+1−Δi+1 of fluid, either according to a composition of a total fluid, or of a least dense phase, or of a mixture of each phase in proportions determined according to values taken by a property calculated for the fluid, including viscosity;
f. storing an amount and composition of fluid subtracted from the system to set a composition acquired at the age ti+1; and
g. a new composition of the system accounted for geochemical modelling upon passage to the next age (ti+1→ti+2).

26. A method as claimed in claim 20, wherein variations in amounts of minerals and fluid constituents exchanged during the sequence of reactions are determined by a process comprising:
a. identifying a stable system for an age ti+1 from a composition at an age ti;
b. identifying mineral reactions causing change from a stable system for the age ti to a stable system for an age ti+1;
c. carrying out a calculation of quantitative balance, by mass and/or in number of moles, of the exchanges operated by the reactions;
d. carrying out a calculation of quantitative balance, by volume, of the exchanges by involving:
i. a thermodynamic database for the minerals,
ii. an equation of state allowing a composition and a density of each phase of the fluid to be calculated;
e. comparing volume variations obtained by geochemical modelling δi+1 and by basin modelling Δi+1 respectively if δi+1 exceeds Δi+1 by an amount determined with regard to an expected precision of fluid balances in a basin model, the composition of the system being modified by removing a volume δi+1−Δi+1 of fluid, either according to a composition of a total fluid, or of a least dense phase, or of a mixture of each phase in proportions determined according to values taken by a property calculated for the fluid, including viscosity;
f. storing an amount and composition of fluid subtracted from the system to set a composition acquired at the age ti+1; and
g. a new composition of the system accounted for geochemical modelling upon passage to the next age (ti+1→ti+2).

27. A method as claimed in claim 21, wherein a volume variation obtained by geochemical modelling comprises:
δᵢ₊₁={(Vₘ)ᵢ₊₁+(V_{f})ᵢ₊₁}−{(Vₘ)ᵢ+(V_{f})ᵢ},
where (Vf)i and (Vm)i respectively represent a volume of fluid and a volume of minerals for the age ti; and
a volume variation obtained by basin modelling comprises:
Δi+1=Vi+1−Vi,
where Vi represents a cell volume obtained by modelling.

28. A method as claimed in claim 22, wherein a volume variation obtained by geochemical modelling comprises:
δᵢ₊₁={(Vₘ)ᵢ₊₁+(V_{f})ᵢ₊₁}−{(Vₘ)ᵢ+(V_{f})ᵢ},
where (Vf)i and (Vm)i respectively represent a volume of fluid and a volume of minerals for the age ti; and
a volume variation obtained by basin modelling comprises:
Δi+1=Vi+1−Vi,
where Vi represents a cell volume obtained by modelling.

29. A method as claimed in claim 23, wherein a volume variation obtained by geochemical modelling comprises:
δᵢ₊₁={(Vₘ)ᵢ₊₁+(V_{f})ᵢ₊₁}−{(Vₘ)ᵢ+(V_{f})ᵢ},
where (Vf)i and (Vm)i respectively represent a volume of fluid and a volume of minerals for the age ti; and
a volume variation obtained by basin modelling comprises:
Δi+1=Vi+1−Vi,
where Vi represents a cell volume obtained by modelling.

30. A method as claimed in claim 24, wherein a volume variation obtained by geochemical modelling comprises:
δᵢ₊₁={(Vₘ)ᵢ₊₁+(V_{f})ᵢ₊₁}−{(Vₘ)ᵢ+(V_{f})ᵢ},
where (Vf)i and (Vm)i respectively represent a volume of fluid and a volume of minerals for the age ti; and
a volume variation obtained by basin modelling comprises:
Δi+1=Vi+1−Vi,
where Vi represents a cell volume obtained by modelling.

31. A method as claimed in claim 25, wherein a volume variation obtained by geochemical modelling comprises:
δᵢ₊₁={(Vₘ)ᵢ₊₁+(V_{f})ᵢ₊₁}−{(Vₘ)ᵢ+(V_{f})ᵢ},
where (Vf)i and (Vm)i respectively represent a volume of fluid and a volume of minerals for the age ti; and
a volume variation obtained by basin modelling comprises:
Δi+1=Vi+1−Vi,
where Vi represents a cell volume obtained by modelling.

32. A method as claimed in claim 26, wherein a volume variation obtained by geochemical modelling comprises:
δᵢ₊₁={(Vₘ)ᵢ₊₁+(V_{f})ᵢ₊₁}−{(Vₘ)ᵢ+(V_{f})ᵢ},
where (Vf)i and (Vm)i respectively represent a volume of fluid and a volume of minerals for the age ti; and
a volume variation obtained by basin modelling comprises:
Δi+1=Vi+1−Vi,
where Vi represents a cell volume obtained by modelling.

33. A method as claimed in claim 9, wherein the fluids comprise one of water, carbon dioxide, hydrocarbon gas, hydrogen, nitrogen or hydrogen sulfide content.

34. A method as claimed in claim 10, wherein the fluids comprise one of water, carbon dioxide, hydrocarbon gas, hydrogen, nitrogen or hydrogen sulfide content.

35. A method as claimed in claim 11, wherein the fluids comprise one of water, carbon dioxide, hydrocarbon gas, hydrogen, nitrogen or hydrogen sulfide content.

36. A method as claimed in claim 13, wherein the fluids comprise one of water, carbon dioxide, hydrocarbon gas, hydrogen, nitrogen or hydrogen sulfide content.

37. A method as claimed in claim 21, wherein the fluids comprise one of water, carbon dioxide, hydrocarbon gas, hydrogen, nitrogen or hydrogen sulfide content.

38. A method as claimed in claim 27, wherein the fluids comprise one of water, carbon dioxide, hydrocarbon gas, hydrogen, nitrogen or hydrogen sulfide content.
